(19) United States
(12) Patent Application Publication
(10) Pub. No.: US 2014/0243428 A1
(43) Pub. Date: Aug. 28, 2014

(54) SELF HEALING HYDROGELS

(71) Applicant: The Regents of the University of California, Oakland, CA (US)

(72) Inventors: Shyni Varghese, San Diego, CA (US); Ameya Phadke, San Diego, CA (US)

(21) Appl. No.: 14/187,006

(22) Filed: Feb. 21, 2014

Related U.S. Application Data
(60) Provisional application No. 61/768,891, filed on Feb. 25, 2013.

(51) Int. Cl. C08L 35/00; A61L 24/00; C09D 135/00; A61K 47/32; A61L 24/06

(52) U.S. Cl. CPC C08L 35/00; A61K 47/32; A61L 24/06; C09D 135/00; A61L 24/0031
USPC 514/772.6; 524/813; 523/118

(57) ABSTRACT

The disclosure provides for self-healing hydrogels, complex structures made therefrom, and use thereof, including use of the hydrogels as self-healing coatings, self-healing sealants, tissue adhesives, and drug carriers.

### CROSS REFERENCE TO RELATED APPLICATIONS

This application claims priority under 35 U.S.C. §119 from Provisional Application Ser. No. 61/768,891, filed Feb. 25, 2013, the disclosure of which is incorporated herein by reference.

### TECHNICAL FIELD

The disclosure provides for self-healing chemically cross-linked hydrogels.

### BACKGROUND

Self-healing has been demonstrated in linear polymers, supramolecular networks, dendrimer-clay systems, metal ion-polymer systems, and multicomponent systems. However, self-healing of permanently cross-linked systems, such as hydrogels, has remained elusive.

### SUMMARY

The disclosure provides for hydrogels that are self-healing. In a further embodiment, hydrogels of the disclosure are chemically cross-linked systems that comprise pendant side chains which have an optimal balance of hydrophilic and hydrophobic moieties. In further embodiment, hydrogels of the disclosure are chemically cross-linked systems comprising N-acryloyl 6-aminocaproic acid based pendant side chains. The disclosure further provides for the use of self-healing hydrogels disclosed herein in a wide variety of devices and/or applications in biology, medicine, and engineering.

In a particular embodiment, the disclosure provides for a self-healing hydrogel that comprises one or more cross-linking precursors and one or more polymer precursors comprising a pendant side-chain of Formula I:

wherein,

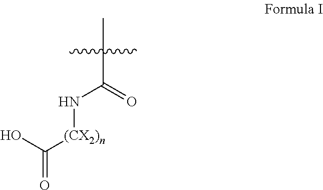

n is an integer from 1 to 10;

each X is independently selected from H, D, optionally substituted (C₁₋₆)-alkyl, optionally substituted (C₁₋₆)-heteroalkyl, optionally substituted (C₁₋₆)-alkenyl, optionally substituted (C₁₋₆)-heteroalkenyl, optionally substituted (C₁₋₆)-alkynyl, optionally substituted (C₁₋₆)-heteroalkynyl, optionally substituted cylcoalkyl, optionally substituted cycicoalkenyl, halide, alcohol, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester. In a certain embodiment, a self-healing hydrogel disclosed herein comprises one or more precursors comprising a pendant side-chain of Formula I, wherein, n is an integer from 1 to 11; and each X is independently selected from H, D, and optionally substituted (C₁₋₆)-alkyl. In a further embodiment, a self-healing hydrogel disclosed herein comprises one or more precursors comprising a pendant side-chain of Formula I(a),

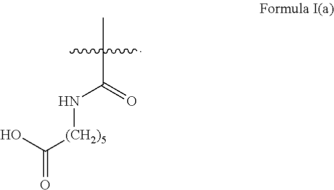

In a particular embodiment, the disclosure provides for one or more cross-linking precursors selected from the group comprising optionally substituted N,N′-methylenebisacrylamide, 1,4-cyclohexanedimethanol divinyl ether, ethylene glycol diacrylate, ethylene glycol dimethacrylate, divinylbenzene, 4,4′-methylenebis(cyclohexyl isocyanate), 1,6-hexanediol diacrylate, 1,4-phenylenediacryloyl chloride, and tetra(ethylene glycol) diacrylate. In a further embodiment, the cross linking precursor is N,N′-methylenebisacrylamide. In another embodiment, a self-healing hydrogel disclosed herein comprises in the range of 0.01% to 1% of cross-linking precursors. In yet another embodiment, a self-healing hydrogel disclosed herein comprises about 0.1% of cross-linking precursors.

In a certain embodiment, the disclosure provides for a self-healing hydrogel disclosed herein, wherein the pendant side chain can form at least two hydrogen bonds to one or more additional pendant side chains. In a further embodiment, the hydrogen bonds can form when hydrogel disclosed herein is exposed to a pH of less than or equal to 5. In yet a further embodiment, the hydrogen bonds break when a hydrogel disclosed herein is exposed to a pH of greater than or equal to 9.

In a particular embodiment, the disclosure provides for structures comprising at least two or more hydrogels disclosed herein that are linked together by hydrogen bonding.

In a certain embodiment, the disclosure provides for a self-healing coating or a self-healing sealant comprising a hydrogel of the disclosure. In another embodiment, the disclosure provides for a tissue adhesive comprising a hydrogel disclosed herein. For example, the tissue adhesive can be used as a mucoadhesive for gastric tissue. In yet another embodiment, the disclosure provides for a drug carrier comprising a hydrogel disclosed herein, such as a drug carrier that controllable releases one or more pharmaceutical agents in the gastrointestinal tract of a subject.

### DESCRIPTION OF DRAWINGS

FIG. 1A-D presents self-healing hydrogels of the disclosure. (A) Schematic illustration of the structure of self-healing A6ACA hydrogels containing dangling side chains terminating with a carboxyl group. (B) Deprotonated cylindrical hydrogels at pH 7.4 (Left) heal in low-pH solution (pH≦3) (Right). In order to more easily distinguish the interface, the hydrogels were dyed yellow and maroon. (C) Healed hydrogels carrying their own weight(s) (Left) and being stretched manually (Right) illustrate the weld-line strength. (D) The healed hydrogels at low pH (Left) separate after exposure to a high-pH solution (with pH>9) (Right). The change in color is due to the reaction of the dyes with the NaOH solution. (Lower) The separated hydrogels in (Upper) repeal upon exposure to an acidic solution (pH<3).

FIG. 2 provides healed hydrogels (Left) that were separated upon exposure to a 30% (wt/vol) solution of urea (Right).

FIG. 3A-C presents the synthesis and characterization of hydrogels of the disclosure. Equilibrium swollen, loosely cross-linked A6ACA hydrogel (A) and its ¹³C NMR spectroscopic analysis (B). The top spectrum represents linear polyA6ACA, whereas the bottom spectrum represents the cross-linked A6ACA. All peaks of cross-linked A6ACA (178.2, 36.1, 33.0, 24.9, 22.5, and 21.2 ppm) are either identical or close to those of linear polymers, except for one peak at 67.0 ppm (labeled by the asterisk). This peak is attributed to chemical cross-links formed during the polymerization as a consequence of chain transfer at high monomer concentration. (C) Swelling ratio measurements for hydrogels synthesized with varying N,N′-methylenebisacrylamide content.

FIG. 4A-E provides a mechanism for the self-healing of hydrogels of the disclosure. Raman (A) and FTIR-ATR (B) spectroscopy of healed (low pH) and unhealed (high pH) hydrogels demonstrating the presence of multiple types of hydrogen-bonded carboxyl groups. (C) Deduced molecular structures of pendant side chains in the faceon and interleaved hydrogen-bonding configurations responsible for the healing at low pH. (D) Structure of the pendant side chains in the unhealed hydrogels at high pH. At high pH, the carboxyl groups become deprotonated, leading to strong electrostatic repulsion between the opposing side chains, thus preventing healing. (E) Schematic explanation for why the healed hydrogels exhibit a mechanically stronger weld line compared to the bulk after healing for small timescales, and vice versa at very long times. Darker gray represents the toughened regions of the hydrogels due to protonation. The lighter gray represents the deprotonated (softer) regions of the hydrogels, which protonates and toughens with increasing exposure to low-pH solution.

FIGS. 5A-B provides molecular modeling to determine steric feasibility of “interleaved” and “face-on” hydrogen bonding configurations of the pendant side chains. (A) Chemical structure of five-unit oligomer of A6ACA (Top) and its energy-minimized structure (Bottom). (B) The energy-minimized structure of two five-unit oligomers facing each other (Top) showing both interleaved (Bottom, Left) and face-on configurations (Bottom, Right). Dotted lines, intermolecular hydrogen bonds; green, alkyl backbone; gray, CH₂ groups in pendant side chain; blue, nitrogen; red, oxygen; and white, polar hydrogen.

FIG. 6A-C provides a characterization of healing and healed hydrogels of the disclosure. (A) Effect of healing time on fracture stress. (B) Stress-strain curve, comparing tensile properties of 24-hour healed gels with a single, unhealed hydrogel at identical conditions. The solid and dashed lines represent data from healed and unhealed hydrogels, respectively. (C) Fracture stress as a function of the extent of cross-linking for hydrogels containing 0.1%, 0.2%, and 0.5% of cross-linker (N,N′-methylenebisacrylamide) content. Error bars in A and C represent the standard deviation (n=3).

FIGS. 7A-B presents fracturing and self-healing dynamics of hydrogels of the disclosure. (A) Hydrogels healed for 5 minutes fracture within the bulk region and not at the weld line. (B) Hydrogels healed for 24 hours, which exhibit an opaque color, fracture at the weld line during tensile testing. The arrows indicate the weld line.

FIGS. 8A-B presents the effect of side-chain length on healing efficiency. (A) Chemical structure of N-acryloyl modified amino acids used to investigate the effect of side-chain length on healing ability (from left to right) N-acryloyl glycine (1 CH₂ group), N-acryloyl 4-aminobutyric acid (3 CH₂ groups), N-acryloyl 6-aminocaproic acid (5 CH₂ groups), N-acryloyl 8-aminocaprylic acid (7 CH₂ groups), and N-acryloyl 11-aminoundecanoic acid (10 CH₂ groups). (B) Table summarizing the effect of pendant side-chain length on healing.

FIG. 9A-D demonstrates the effect of side-chain length on the accessibility of functional groups. (A) Solubility of carboxylic acids of varying hydrocarbon chain lengths in water (black circles). Dashed red line indicates the density of carboxyl groups present in the hydrogels. (B) Molecular dynamics simulations setup for A6ACA network. A nine-arm motif of the network (Left) is used to create the 3D network structure (Right) via periodic boundary conditions. (C) Computed accessibilities of the amide and carboxyl groups in the A6ACA, A8ACA, and A11AUA hydrogels. (D) Representative configuration of the A6ACA and A11AUA network obtained from molecular dynamics simulations, shown in terms of solvent excluded surface, illustrating the higher accessibility of the amide groups in the former network. Blue, red, light gray and white colors correspond to the surfaces of nitrogen, oxygen, carbon, and hydrogen, respectively. Chain length n in A and C represent number of CH₂ groups in the carboxylic acids and side chains, respectively.

FIG. 10A-F provides applications of self-healing hydrogels of the disclosure. The rupture site within the A6ACA coating on a polystyrene surface (A) before and (B) after healing. The coating was colored using a dye for easy visualization and the observed color change after healing is caused by its exposure to low-pH buffer. (Scale bars: 500 μm.) (C) Adhesion of A6ACA hydrogels to a poly(propylene) surface. (D) Polypropylene container holding acid solution after sealing the hole with A6ACA hydrogel. The arrow indicates the sealed site. (E) Adhesion of A6ACA hydrogels to rabbit gastric mucosa. (F) Cumulative tetracycline release from A6ACA hydrogels plotted as a function of time. Error bars represent standard deviation (n=4).

### DETAILED DESCRIPTION

As used herein and in the appended claims, the singular forms “a,” “and,” and “the” include plural referents unless the context clearly dictates otherwise. Thus, for example, reference to “pendant side chain” includes a plurality of such pendant side chains and reference to “hydrogel” includes reference to one or more hydrogels and equivalents thereof known to those skilled in the art, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in their entirety for the purposes of describing and disclosing methodologies, which are described in the publications that might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

There is increasing interest in the development of “smart” materials that can sense changes in their environment and can accordingly adapt their properties and function, similar to living systems. Over the last decade, smart hydrogels have been developed that exhibit unique bio-mimicking functions: thermo-responsive volume phase transitions similar to sea cucumbers, self-organization into core-shell hollow structures similar to coconuts, shape memory as exhibited by living organisms, and metal ion-mediated cementing similar to marine mussels. So far, self-healing has been demonstrated in linear polymers, supramolecular networks, dendrimer-clay systems, metal ion-polymer systems, and multicomponent systems. Whereas multicomponent thermosetting systems harness the ability of embedded chemical agents to repair cracks, supramolecular networks and noncovalent hydrogels employ secondary interactions such as hydrogen bonding, ionic interactions, and hydrophobic association for healing. In spite of the many applications in biomedical sciences that such aqueous healing systems could offer, self-healing of permanently cross-linked systems such as hydrogels has remained elusive because of the presence of water and irreversible chemical cross-links. Accordingly, there has been a long felt need in the industry for the development of permanently cross-linked materials, such as hydrogels, which can perform autonomous healing upon damage.

The disclosure provides for hydrogels that are capable of self-healing. In studies presented herein, it was found that hydrogels could undergo self-healing by decorating the polymer network with dangling hydrocarbon side chains containing polar functional groups that would mediate hydrogen bonding across two separate hydrogel pieces or across a rupture in the hydrogel. In a particular embodiment, the disclosure provides for robust and efficient healing of the hydrogels disclosed herein by ensuring that the functional groups across the interface are accessible to each other beyond the corrugation of the interface. In a further embodiment, the hydrogels of the disclosure comprise side chains of a suitable length so that the overall network is sufficiently deformable. In yet a further embodiment, the side chains are of a length so that: (1) there is a certain level of flexibility, (2) steric hindrance of the interacting functional groups is minimized, and/or (3) to prevent hydrophobic collapse of the side chains. In another embodiment, the disclosure provides for hydrogels which comprise side chains that possess a balance of hydrophobic and hydrophilic moieties.

In further studies presented herein, hydrogels which were synthesized from polymer precursors comprising A6ACA side chains exhibited self-healing in an aqueous environment in spite of the hydrogels irreversible cross-linked architecture. In a certain embodiment, the disclosure provides for hydrogels which are comprised of polymer precursors which comprise acryloyl-6-aminocaproic acid (A6ACA) based side chains.

Scheme I and Scheme II are presented herein which enable the synthesis of self-healing hydrogels of the disclosure. It should be understood, however, that obvious modifications can be made to the following schemes, such as performing steps in the presence of catalysts; use of alternative solvents/solvent systems, bases, and acids; substitution of alternate cross-linking precursors; performing the reaction steps at elevated temperatures; and incorporating purification steps (e.g., extractions, dialysis, recrystallizations, and column chromatography). Accordingly, the following Schemes are presented as a general guide to synthesize hydrogels of the disclosure and it can be further expected that one of ordinary skill in the art can make obvious substitutions to one or more reaction steps presented in Scheme I and/or Scheme II.

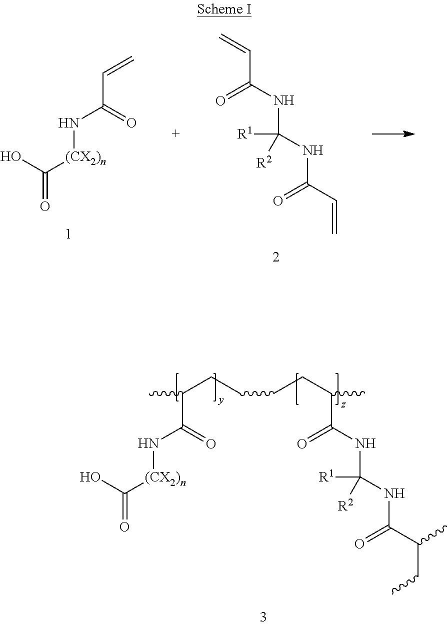

Compound 1 and compound 2 are polymerized and the polymers are cross-linked by adding an appropriate radical initiator, such as 0.5% ammonium persulfate, and an appropriate radical accelerator, such as 0.1% N,N,N′,N′-tetramethylethylene dimaine, at an elevated temperature to afford hydrogel 3. In particular, the amount of cross linking of hydrogel 3 can be controlled by varying the amount of compound 2 with respect to compound 1. Moreover, alternate crosslinking precursors can be substituted for compound 2.

In an alternate embodiment, a loosely cross-linked hydrogel can made according to Scheme II.

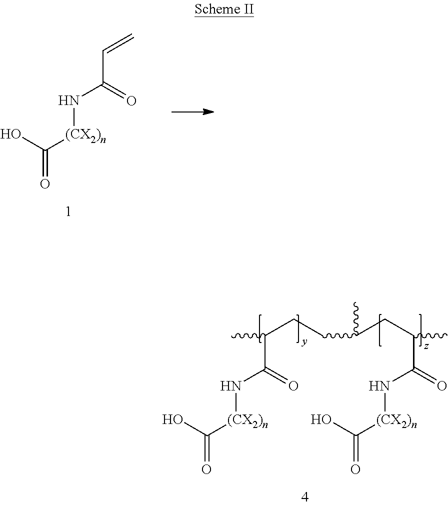

Via chain transfer, high concentrations of compound 1 in the presence of an appropriate base, such as sodium hydroxide, is polymerized by adding an appropriate radical initiator, such as 0.5% ammonium persulfate, and an appropriate radical accelerator, such as 0.1% N,N,N′,N′-tetramethylethylene dimaine, at an elevated temperature to form hydrogel 4.

In a certain embodiment, a self-healing hydrogel disclosed herein comprises a plurality of polymer monomers which comprise a pendant side-chain of Formula I:

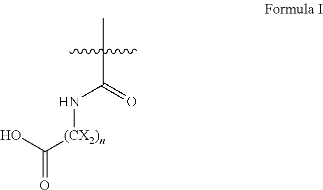

wherein,

n is an integer from 1 to 10;

each X is independently selected from H, D, optionally substituted (C₁₋₆)-alkyl, optionally substituted (C₁₋₆)-heteroalkyl, optionally substituted (C₁₋₆)-alkenyl, optionally substituted (C₁₋₆)-heteroalkenyl, optionally substituted (C₁₋₆)-alkynyl, optionally substituted (C₁₋₆)-heteroalkynyl, optionally substituted cylcoalkyl, optionally substituted cycicoalkenyl, halide, optionally substituted oxygen containing functional group (e.g., alcohol, ketone, aldehyde, acyl halide, carbonate, carboxylic acid, ester, and ether), optionally substituted nitrogen containing functional group (e.g., amide, amine, imine, azide, cyanate, azo, nitrate, nitrile, nitro, and nitroso), optionally substituted sulfur containing functional group (e.g., thiol, sulfide, disulfide, sulfoxide, sulfone, sulfinic acid, sulfonic acid, thiocyanate, thione, and thial), optionally substituted phosphorous containing functional group (e.g., phosphine, phosphonic acid, phosphate, phosphodiester), optionally substituted boron containing functional group (e.g., boronic acid, boronic ester, borinic acid, and borinic ester).

In a further embodiment, a self-healing hydrogel disclosed herein comprises a plurality of polymer monomers which comprise a pendant side-chain of Formula I:

wherein,

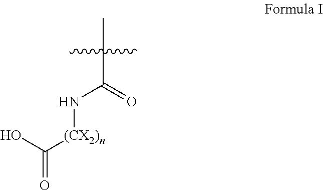

n is an integer from 4 to 8;

each X is independently selected from H, D, and an optionally substituted (C₁₋₆)-alkyl.

In yet a further embodiment, a self-healing hydrogel disclosed herein comprises a plurality of monomers which comprise a pendant side-chain of Formula I(a):

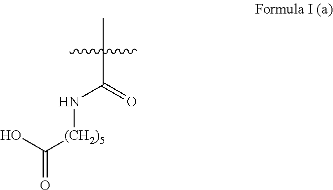

The disclosure further provides that the properties of the hydrogels of the disclosure can be influenced by a variety of factors including, but not limited to, cross-linking density, side-chain length, and the hydrophobicity/hydrophilicity of side-chain moieties.

In a particular embodiment, the self-healing of hydrogels disclosed herein can be controlled by controlling the extent of cross-linking in the hydrogel. In a further embodiment, the extent of cross-linking a hydrogel disclosed herein can be controlled by the varying the percentage of a cross-linking precursor, such as N,N′-methylenebisacrylamide. In a further embodiment, hydrogels of the disclosure comprise a minor percentage of cross-linking precursors. In another embodiment, hydrogels of the disclosure comprise from 0.01% to 1% of cross-linking precursors. In yet another embodiment, hydrogels disclosed herein comprise about 0.1% of N,N′-methylenebisacrylamide. Examples of additional cross-linker precursors that can be used with the hydrogels disclosed herein, include optionally substituted N,N′-methylenebisacrylamide, 1,4-cyclohexanedimethanol divinyl ether, ethylene glycol diacrylate, ethylene glycol dimethacrylate, divinylbenzene, 4,4′-methylenebis(cyclohexyl isocyanate), 1,6-hexanediol diacrylate, 1,4-phenylenediacryloyl chloride, and tetra(ethylene glycol) diacrylate.

In a certain embodiment, the disclosure further provides that the self-healing of hydrogels disclosed herein can be controlled by varying the pendant side chain lengths (i.e., for Formula I the integer specified for n). In a further embodiment, hydrogels disclosed herein have pendant side chains comprising between 4 to 8 methylene groups. In yet further embodiments, hydrogels disclosed herein have pendant side chains comprising 5 methylene groups. In another embodiment, the self-healing of hydrogels disclosed herein can be influenced, positively or negatively, by substituting a methylene group with a different functional group, such as an optionally substituted (C₁₋₆) alkyl.

In a particular embodiment, the disclosure provides that the self-healing of hydrogels disclosed herein can be controlled by forming one or more hydrogen bonds between moieties of pendant side chains. It should also be understood that the disclosure provides for a wide variety of functional groups in addition to methylene groups provided for in the Examples. These functional groups may contain moieties that can form hydrogen bonds under certain conditions (i.e., X of Formula I can be an amine, hydroxyl, alkyl halide, and thiol based functional groups). Accordingly, three, four, five or more hydrogen bonds can be expected to form between moieties of pendant side chains which contain these hydrogen bond donor and/or hydrogen bond acceptor moieties. In a certain embodiment, a carboxylic acid group from a pendant side chain of hydrogel disclosed herein can form two hydrogen bonds with carboxylic acid groups from other pendant side chains. In an alternate embodiment, a carboxylic acid group can form two hydrogen bonds with amide groups from other pendant side chains. In another embodiment, one or more hydrogen bonds can form between pendant side chains in a “face on” configuration and/or interleaved configuration when a hydrogel disclosed herein is exposed to a pH less than or equal to 3. In yet a further embodiment, one or more hydrogen bonds between pendant side chains are broken when a hydrogel disclosed herein is exposed to a pH that is greater than or equal to 9. In a particular embodiment, hydrogels disclosed herein can heal or re-heal by forming one or more hydrogen bonds between pendant side chains when the hydrogel is exposed to a pH less than or equal to 3.

The hydrogels of the disclosure remained healed over a wide range of temperatures, light conditions, and humidity. Accordingly the hydrogels of the disclosure have numerous applications in medicine, environmental science, and industry. Non-limiting representative applications are presented herewith. For example, hydrogels of the disclosure as self-repairing coatings and sealants were tested. Various surfaces were coated with hydrogels disclosed herein and mechanically damaged by introducing 300-μm-wide cracks (see FIG. 10A). The coatings healed the imparted crack within seconds upon exposure to low-pH buffers (see FIG. 10B). Because this healing only required initial contact, one can achieve repair by simply spraying the cracks with a low-pH buffer. It was also found that hydrogels of the disclosure could adhere to various plastics like polypropylene and polystyrene even in their hydrated state; this is likely due to hydrophobic interactions (see FIG. 10C). This finding, in conjunction with the observed rapid pH-dependent healing, suggests that the hydrogels of the disclosure could be used as sealants for vessels containing corrosive acids. As a proof-of-concept, a hole was created in a polypropylene container that was then coated with a hydrogel disclosed herein. When hydrochloric acid was poured into the container, the hydrogel instantly sealed the hole and prevented any leakage of the acid (see FIG. 10D). The use of hydrogels disclosed herein as a tissue adhesive was also investigated by using fresh gastric mucosa of rabbits. Hydrogels of the disclosure adhered well to the gastric mucosa and that the adhesion was strong enough to support the weight of the hydrogel (see FIG. 10E). Hydrogels disclosed herein, therefore could be used as tissue adhesives for stomach perforations, where the lightly cross-linked hydrogels could be injected to prevent leakage of gastric acids. In further studies presented herein, tetracycline loaded hydrogels were exposed to a simulated gastric acid environment (pH 1.5) and the drug-release profile was evaluated. Tetracycline was released at a constant rate for 4 days after the initial bolus release (FIG. 10F). Accordingly, hydrogels disclosed herein could also be employed for drug delivery, by storing and releasing bioactive molecules without compromising their carriers. The ability of hydrogels disclosed herein to fuse together could also allow for the development of soft structures with complex architectures. Such structures could find applications as soft actuators and in robotic devices.

The following examples are intended to illustrate but not limit the disclosure. While they are typical of those that might be used, other procedures known to those skilled in the art may alternatively be used.

### EXAMPLES

### Monomer Synthesis and Characterization

Monomers N-acryloyl 2-glycine (A2AGA), N-acryloyl 4-aminobutyric acid (A4ABA), N-acryloyl 6-aminocaproic acid (A6ACA), N-acryloyl 8-aminocaprylic acid (A8ACA), and N-acryloyl 11-aminoundecanoic acid (A11AUA) were synthesized from glycine (Fisher Scientific, Inc.), 4-aminobutyric acid, 6-aminocaproic acid, 8-aminocaprylic acid (Acros Organics, Inc.), and 11-aminoundecanoic acid (Aldrich), respectively, as is described in Ayala, et al., Biomaterials (2011) 32:3700-3711, which is incorporated herein in its entirety.

Briefly, for A2AGA, glycine (0.1 mol) and NaOH (0.11 mol) were dissolved in deionized water (80 mL) in ice bath under vigorous stirring. To this, acryloyl chloride (0.11-mol) in tetrahydrofuran (15 mL) was added dropwise. The pH was maintained at 7.5-7.8 until the reaction was complete. The reaction mixture was then extracted with ethyl acetate. The clear aqueous layer was acidified to pH 2.0 and then extracted again with ethyl acetate. The organic layers were collected, combined, and dried over sodium sulfate. The solution was then filtered, concentrated, and precipitated in petroleum ether. Further purification was achieved by repeated precipitation and the product was lyophilized. Synthesis of other monomers followed similar procedure, with variations in pH during the acidification: pH 2.0 for A4ABA, pH 3.0 for A6ACA, and pH 5.0 for A8ACA and A11AUA. Proton nuclear magnetic resonance spectra (¹H NMR) of monomers were recorded with a Varian Mercury-400 spectrometer at 400 MHz. Carbon-13 nuclear magnetic resonance spectra (¹³C NMR) were recorded on a Varian Mercury-400 spectrometer at 100 MHz; CDCl₃ or D₂O were used as solvents.

Synthesis of Hydrogels.

Hydrogels were prepared by free radical polymerization in aqueous solution containing 1 mmol/mL of monomer, N,N′-methylene bisacrylamide (Bis-Am), 0.5% ammonium persulfate (initiator), and 0.1% tetramethylethylenediamine (accelerator).

### Synthesis of N-acryoloyl amino acid Bis-Am hydrogels with varying side-chain lengths (y>>z)

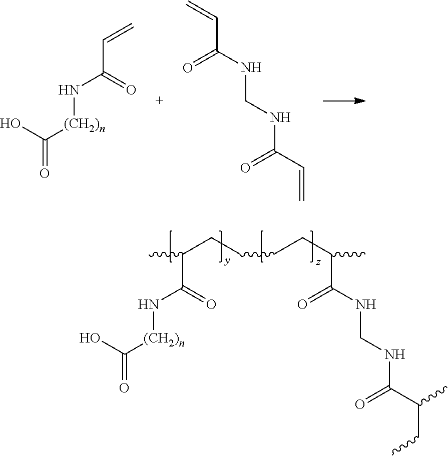

To synthesize A6ACA hydrogels containing different cross-linker (N,N′-methylenebisacrylamide) content, 0.1%, 0.2%, and 0.5% (wt/vol) BisAm (Sigma-Aldrich, Inc.) was added to the 1 M deprotonated A6ACA solution and polymerized as described above using the ammonium persulfate/tetramethylethylenediamine (APS/TEMED) redox initiators for 16 hours at 37° C. To create hydrogels with varying pendant side chains, we followed the same procedure. Specifically, 1 M solutions of the respective monomers 0.1291 g/mL for A2AGA (n=1), 0.157 g/mL for A4ABA (n=3), 0.185 g/mL for A6ACA (n=5), 0.213 g/mL for A8ACA (n=7), and 0.241 g/mL for A11AUA (n=10) were deprotonated using equimolar NaOH and used.

### Synthesis of loosely cross-linked N-acryoloyl 6-aminocaproic hydrogels (y>>z)

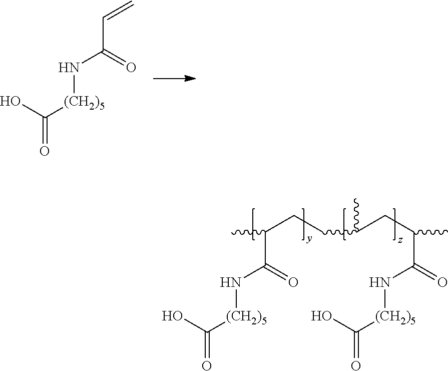

Loosely cross-linked A6ACA hydrogels were prepared using high concentrations of A6ACA monomers via chain transfer. 1 M A6ACA was dissolved in 1 M sodium hydroxide to deprotonate the carboxyl groups of A6ACA. This solution was then polymerized using 0.5% APS as initiator and 0.15% TEMED as accelerator in cylindrical polypropylene molds measuring 0.5 cm in diameter and 2.5 cm in length. Polymerization was allowed to proceed for 16 hours at 37° C.

Synthesis of Linear A6ACA Polymer.

Two grams (10.8 mmol) of A6ACA, 0.432 g (10.8 mmol) of NaOH, and 7.8 mg (0.1 mmol) of 2-mercaptoethanol (chain transfer agent) were dissolved in DI water (40 mL) at room temperature. Upon complete dissolution of the reactants, TEMED (40 μL) was added to the solution, and purged with argon for 30 minutes; APS (20 mg) in DI water (2 mL) was then added to the solution under argon purge. The solution was transferred to an oil bath at 40° C. and reacted overnight. The polymer solution was cooled to room temperature and poured into acetone (800 mL). The precipitate was collected and dried in vacuo at room temperature. The product was further purified by dialysis against DI water in a dialysis tube (MWCO=500 Da) for 48 hours and freeze-dried before analysis. The usage of 2-mercaptoethanol as a chain transfer agent prevented the transfer of free radicals to the polymer backbone, thereby subsequent cross-linking of the polymer was prevented.

Synthesis of Complex Structures Using Healing Ability of A6ACA Hydrogels.

Cylindrical A6ACA hydrogels were swollen in PBS containing 0.5% methyl red or approximately 0.002% alizarin red S, respectively. Using the yellow pieces (swollen in methyl red), hydrogels were healed to form a letter “U” with 0.5 mL HCl. Following this, the healed U was separated into the different pieces using 1 N NaOH. These pieces were then re-healed to form the letter “S.” Using the pieces swollen in the alizarin red S (appearing maroon in color), a similar procedure was carried to form the letters “C” and “D.”

Healing of the Hydrogels.

Healing of hydrogels was carried out in different buffer solutions with pH ranging from 0.3-7.4. Specifically, we used 0.5 M hydrochloric acid (pH 0.3), 1× phosphate-buffered saline (pH 7.4), and other buffer solutions, are provided in Table 1. Table 1 provides the composition of buffers with varying pHs.

TABLE 1

| Buffer | |
|---|---|
| pH | Composition |
| 1 | 27.17% 0.2M potassium chloride + |
| | 72.83% 0.2M hydrochloric acid |
| 3 | 69.15% 0.1M potassium hydrogen phthalate + |
| | 30.85% 0.1M hydrochloric acid |
| 4 | 99.8% 0.1M potassium hydrogen phthalate + |
| | 0.02% 0.1M hydrochloric acid |
| 4.5 | 85.18% 0.1M potassium hydrogen phthalate + |
| | 14.82% 0.1M sodium hydroxide |
| 5 | 68.87% potassium hydrogen phthalate + |
| | 31.13% 0.1M sodium hydroxide |

The hydrogel samples were brought into contact with each other without application of any external force. For ease of visualization, the hydrogels were dyed yellow and maroon by soaking them in PBS containing 0.5% (vol/vol) methyl red indicator and approximately 0.002% (wt/vol) alizarin red S, respectively.

Mechanical Characterization.

Butt-welded hydrogels were used for mechanical measurements. To determine the interfacial strength of hydrogels healed for 10 seconds and 5 minutes, a custom-designed approach was used where known weights were applied to healed hydrogels and the resulting engineering stress required to break the healed hydrogels was calculated. The mechanical properties of 24 hour healed hydrogels were determined using an Instron 3342 Universal Testing System (Instron) equipped with a Model 2519-104 force transducer. A load cell of 450N was fitted to the instrument and the tensile tests were done at a cross-head speed of 15 mm/min. The data acquisition and processing were performed with BlueHill software. The tensile modulus was determined by calculating the slope of a linear region of stress-strain curve, whereas the fracture stress was determined from the peak of the curve.

Reversibility of Healing.

Cylindrical hydrogels were healed via butt welding, as described above, and then immersed in 1 M NaOH (pH 14) for 10 minutes for separation. The separated hydrogels were then briefly rinsed in PBS to remove excess NaOH and reintroduced into an acidic solution (pH 0.3) and healed by maintaining the surfaces in contact for less than 5 seconds. These re-healed hydrogels were then reintroduced into 1 M NaOH solution for separation. This cycle of healing-separation-repealing was performed more than 12 times to test the reversibility of healing. Separation of healed hydrogels was also examined in a standard buffer solution of pH 10 (Fisher Scientific, Inc.), and it was found to be at a slower rate compared to those separated in pH-14 buffer.

Stability of Healed Hydrogels in Water and Effect of Temperature.

The completely healed hydrogels were immersed in deionized (DI) water for more than a month to determine their stability at ambient temperature. To determine the effect of temperature on the stability, the healed hydrogels were immersed in boiling water at 100° C. for 1 hour.

Effect of Urea on Healing Efficacy.

To investigate the contribution of hydrogen bonding on healing, the butt-welded hydrogels were immersed in excess of a 30% (wt-vol) solution of urea in DI water. Another healed hydrogel immersed in DI water was used as the control.

FTIR-ATR and Raman Spectroscopy.

Spectroscopic analysis was carried out on loosely cross-linked A6ACA hydrogels that were healed in 0.5 M HCl for 24 hours, along with unhealed hydrogels (pH approximately 7.4) for comparison. The healed and unhealed hydrogels were dried for 24 hours at 37° C. prior to performing Raman and FTIR-ATR spectroscopy to minimize interference of hydrogen-bonded water molecules. The FTIR spectra from 4,400 to 600 cm⁻¹ were acquired with a Perkin Elmer Spectrum RX Fourier transform infrared spectrometer. Samples were placed on the diamond window of a PIKE MIRacle ATR attachment. Each reported spectrum is the average of four scans, and the resolution is 2 cm⁻¹. Raman spectroscopy was performed with a homebuilt Raman microscope system. A mixed-gas Kr—Ar ion laser (Coherent Innova 70C) provided continuous-wave excitation at 514.5 nm. The beam was sent through a 514.5-nm interference filter (Semrock) and directed into a modified fluorescence alignment port of a Zeiss Axio Imager Alm upright microscope. A broadband beam splitter (Edmund Optics) directed a small portion (approximately 10%) of the beam downward to the entrance aperture of a 50× objective. The power at the sample was 5.2 mW. Back-scattered light was collected and collimated with the same objective, filtered with a 514.5-nm edge filter (Semrock), and focused on the entrance slit of a 0.32-m focal length spectrograph (Horiba Jobin Yvon; iHR-320). Raman scattered light was dispersed with a 1,200 grooves/mm-ruled grating and detected by a thermoelectrically cooled open-electrode CCD detector (Horiba Jobin Yvon Synapse). Wavelength calibration was performed using known lines of Hg/Ar and Ne lamps for windows centered at 550 and 610 nm, respectively.

A6ACA Hydrogels Demonstrate Rapid and Robust Self-Healing:

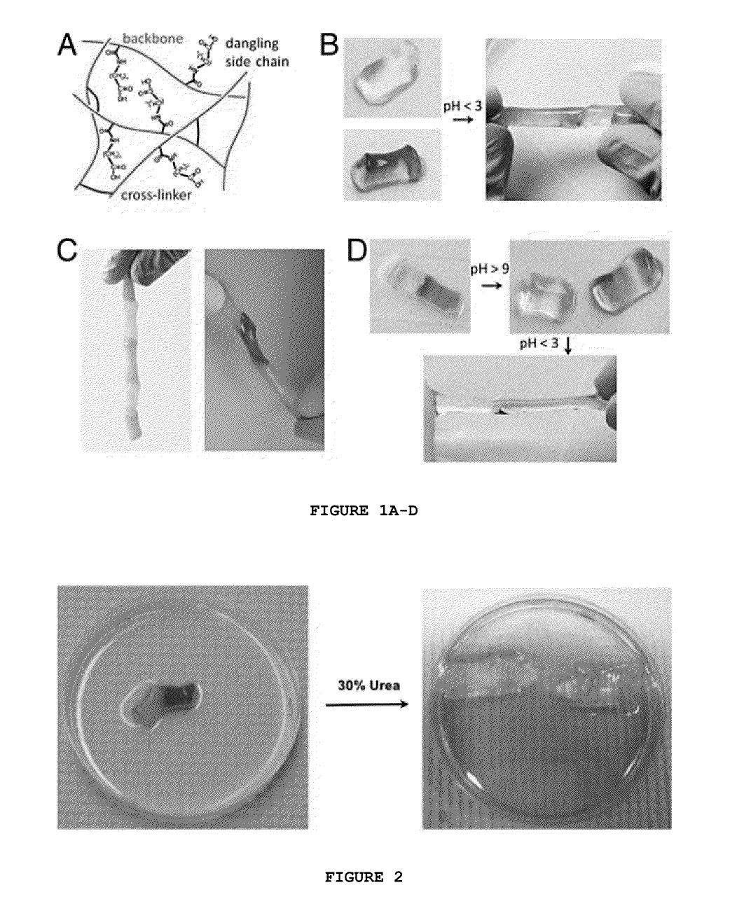

The A6ACA hydrogels were synthesized as described herein. It was observed that two lightly cross-linked A6ACA hydrogels weld rapidly to each other within 2 seconds when brought in contact in low-pH aqueous solution (pH 3) (see FIG. 1B). The healed hydrogels exhibited a strong interface capable of withstanding their own weight(s), repeated stretching, and exposure to boiling water (see FIG. 1C). The healed samples were able to sustain large deformations and still recover their size and shape when the stress was released. This pH-mediated healing was also reversible: Two healed hydrogels separated when exposed to high pH (see FIG. 1D). The separated hydrogels were able to re-heal upon reintroduction into a low-pH environment (see FIG. 1D). The cycle of healing, separation, and re-healing was repeated many (>12) times without hysteresis; the healing occurred on the same timescale and had comparable weld-line strength as that of the original hydrogels.

Hydrogel Swelling Ratio Measurements.

The hydrogels were immersed in excess of 1×PBS (pH 7.4) for 48 hours following synthesis to allow equilibration with constant change of PBS. The hydrogels were weighed after equilibrium swelling to determine their wet weight and after subsequent freeze-drying to determine their dry weight. Swelling ratio was calculated as the ratio of wet to dry weight.

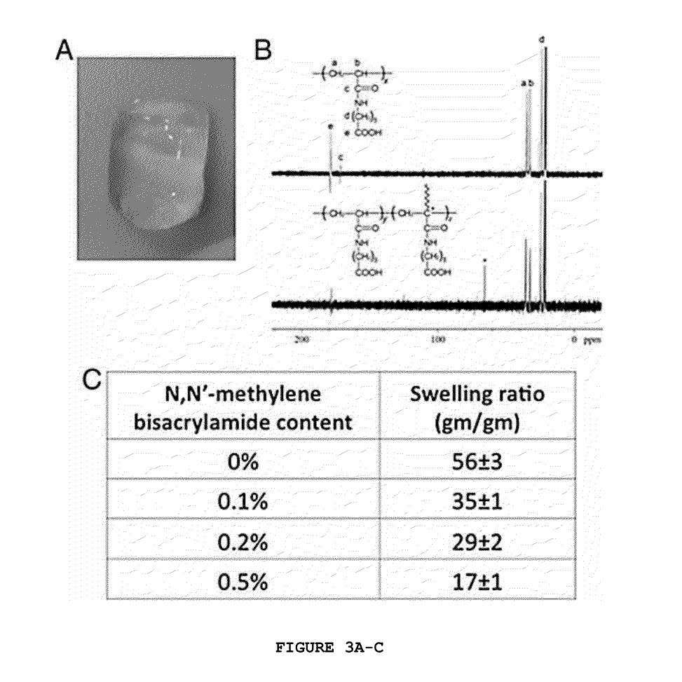

The as-synthesized hydrogels exhibited an intact swollen structure with an equilibrium swelling ratio of 56±3 g/g in PBS (see FIG. 3A). The hydrogel formation was also characterized through ¹³C NMR spectroscopy (see FIG. 38). The cross-linked hydrogels were dialyzed against deionized (DI) water in a dialysis tube [molecular weight cutoff (MWCO)=500 Da] for 48 hours and ground in a mortar into fine particles and then freeze-dried. The freeze-dried powder was swollen in D₂O for NMR analysis. Carbon-13 NMR spectroscopy was recorded on a Varian VX500 500 MHz spectrometer. The NMR spectrum was compared against linear A6ACA polymer as described herein. Linear poly(N-acryloyl 6-aminocaproic acid) was dissolved in D₂O at a concentration of 1% (wt/vol); lightly cross-linked polyA6ACA was fully swollen in D₂O and transferred into an NMR tube before spectroscopic analysis. FIG. 3B shows that all peaks of the loosely crosslinked-A6ACA hydrogels (178.2, 36.1, 33.0, 24.9, 22.5, and 21.2 ppm) were identical or close to those in the spectrum of linear polyA6ACA except for one peak at 67.0 ppm (labeled by an asterisk). This additional peak is attributed to chemical crosslinks formed during polymerization as a consequence of chain transfer at high monomer concentration. Samples were prepared as triplicates and averages were calculated with standard deviation (see FIG. 3C).

Spectroscopic Analyses.

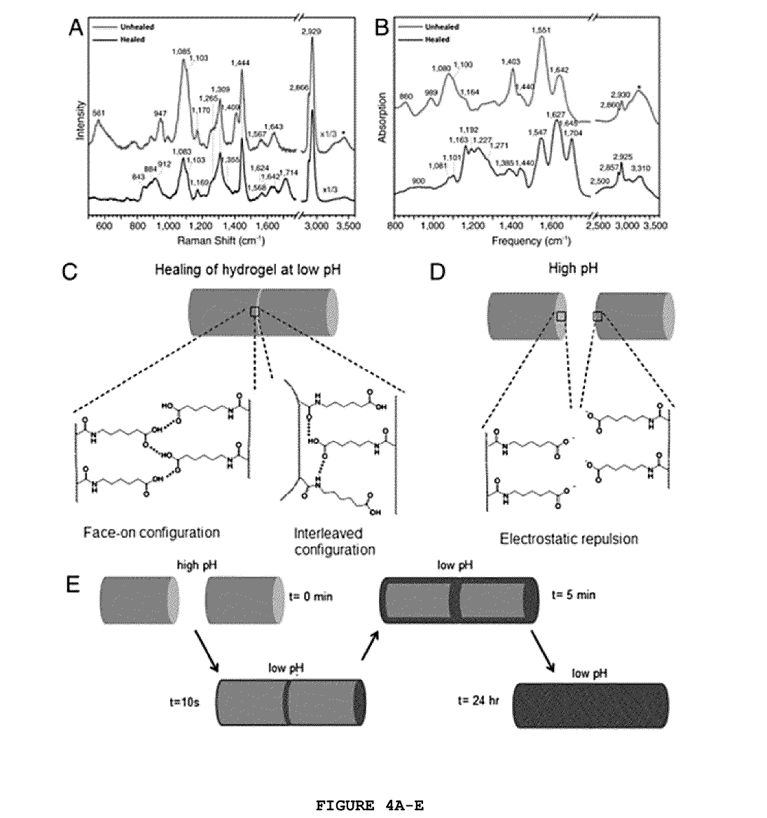

The spectroscopic analyses presented herein were deduced in-part by using Colthup et al. Introduction to Infared and Raman Spectroscopy (Academic, New York), pp 289-325 (1975); Barbucci et al., Markomol Chem (1989) 190:2627-2638; Barth et al., Quart Rev Biophys (2002)35:369-430; and Dong et al., Macromolecules (1997) 30:1111-1117, which disclosures are incorporated herein in their entirety. Spectroscopic analysis was carried out on healed and unhealed A6ACA hydrogels. Samples were dried at 37° C. for 24 hours prior to measurement of Raman and FTIR spectra. The Raman spectra of the healed and unhealed are shown in FIG. 4A. The appearance of a band at 1,409 cm⁻¹ for unhealed hydrogels indicates the presence of carboxylate (COO⁻) functional groups. This frequency is well within the typical range expected for the COO⁻ symmetric stretch; the corresponding band in the FTIR spectrum is at 1,403 cm⁻¹ (see FIG. 4B). The healed samples uniquely exhibit a strong Raman band at 1,714 cm⁻¹ (see FIG. 4A), which is best assigned to a hydrogen-bonded carboxylic acid group. The corresponding band in the FTIR spectrum (see FIG. 4B) is at 1,704 cm⁻¹. The close correspondence of the IR and Raman frequencies indicates that this pair of bands is unlikely to be a signature of a cyclic carboxylic acid dimer. A cyclic dimer generally has a Raman-active in-phase combination of C═O stretches that is expected to be downshifted to 1,680-1,640 cm⁻¹ and an IR active out-of-phase combination located in the 1,720-1,680 cm⁻¹ range. Furthermore, a cyclic of out-of-plane OH . . . O hydrogen deformation in the 960-875 cm⁻¹ region of the FTIR spectrum, and these bands are either weak or absent in healed protonated hydrogels. It was assumed that some of the protonated —COOH groups reach the amide groups of opposing pendant chains, and form a pair of H bonds (acceptor and donor) to the respective NH and C═O groups of the amide (interleaved configuration). The following spectral assignments are consistent with this configuration: (1) Raman and IR activity at 1,714/1,704 cm⁻¹ is attributed to the C═O stretch of the carboxylic acid group that is hydrogen bonded to the NH of the amide; (2) the NH stretch at 3,310 cm⁻¹ is a typical frequency for an H-bonded N—H group; and (3) a prominent band in the healed A6ACA hydrogel FTIR spectrum at 1,627 cm⁻¹ and a corresponding weak band in the Raman spectrum at 1,624 cm⁻¹ are assigned to the amide I mode (majority C═O stretch, some C—N stretch). These frequencies are approximately 18 cm⁻¹ downshifted relative to the 1,642-1,643 cm⁻¹ band that is assigned to the amide I at high pH. A downshift is expected for enhanced H bonding to the amide group such as provided by direct interaction between —COOH and amide groups. A substantial amide I band was observed at an intensity of approximately 1,645 cm⁻¹ (FTIR, shoulder) and 1,642 cm⁻¹ (weak Raman band) for healed hydrogels, which is nearly the same frequency as the amide I in unhealed hydrogels (see FIGS. 4A-B and Table 2). This observation suggests that some population of amide groups has a similar H-bond environment at both pH extremes. For this population of interacting chains, the formation of interleaved pendant chains at low pH seems implausible. Instead, it is possible that some of the —COOH groups hydrogen bond with pairs of —COOH groups on opposing strands, in a manner that has previously been termed as “face-on.” The amide groups of this subset of pendant chains would have similar interactions with each other (or with a neighboring water molecule) at either high or low pH, which is consistent with the similar amide I frequencies at low and high pH. Furthermore, the fact that the face-on structure is less symmetric than a cyclic dimer is consistent with the overlapping IR and Raman frequencies. TABLE 2 presents the observed frequencies and assignments of infrared and Raman bands of healed and unhealed A6ACA hydrogels.

TABLE 2

| Low pH | | High pH | | |
|---|---|---|---|---|
| Raman | IR | Raman | IR | Assignments |
| — | 3,310 | — | — | N—H stretch, H bonded |
| 2,929; 2,866 | 2,925; 2,857 | 2,929; 2,866 | 2,930; 2,860 | C—H stretches |
| — | 2500 (broad) | — | — | Overtone/combination bands |
| 1,714 | 1,704 | — | — | C═O stretch of terminal |
| | | | | carboxylic acid |
| 1,642 | 1,645 | 1,643 | 1,642 | Amide I |
| 1,624 | 1,627 | — | — | Amide I (H bonded to —COOH) |
| 1,568 | 1,547 | 1,567 | 1,551 | Amide II; overlap with COO⁻ |
| | | | | stretch for high-pH IR |
| 1,444 | 1,440 | 1,444 | 1,440 | CH₂ bend deformation |
| — | — | 1,409 | 1,403 | COO⁻ stretch |
| 1,355 | 1,386 | — | — | OH deformation |
| 1,309; 1,265 | — | 1,309; 1,265 | — | CH₂ wag or twist, and Amide III |
| — | 1,271-1,192 | — | — | C—O stretch coupled with |
| | | | | C—O—H in-plane bend |
| 1,169-1,803 | 1,163-1,081 | 1,170-1085 | 1,164-1,080 | C—CH₂ and N—CH₂ stretches |
| 912-843 | 900 (broad) | 947 | 860; 989 | unassigned |
| — | — | 561 | — | COO⁻ rock |

Molecular Dynamics Simulations of Hydrogel Networks.

To investigate the effect of side-chain length on healing efficiency, molecular dynamics simulations were performed with hydrogel networks built from A6ACA, A8ACA, and A11AUA monomers having side chains of lengths 5, 7, and 10 CH₂ groups, respectively. The 3D network structure of the hydrogel comprising of 20 monomers between each cross-link was assembled using the procedure of Jang et al., J Phys Chem B (2009) 113:6604-6612, which is incorporated herein in its entirety. The simulation box (unit cell) had dimensions of approximately 9.2×6.4×6.4 nm, and it consisted of water molecules and a nine-arm network motif placed symmetrically inside the simulation box. This motif consisted of two four-arm crossed junctions connected along the x direction by a chain of 20 monomers, where each arm was a chain of 10 monomers (see FIG. 4A, Left). The motif was placed symmetrically inside the simulation box such that its arms in the y and z direction touched the faces of the simulation box, whereas there was a 0.5-nm gap on either side of the two junctions in the x direction. The simulation box, when replicated in all directions through periodic boundary conditions (PBCs), yielded the desired hydrogel network shown in FIG. 4B (Right). Specifically, implementation of PBCs allowed for the covalent connection of the eight-arm ends to their periodic images in the y and z direction, whereas the water filled gap in the ±x direction prevented continuity of the network along the x direction and allowed the creation of a hydrogel-water interface in between periodic images of the network. Because of computational challenges, high cross-link densities were examined: the cross-links in our network are separated by 20 monomers-long chains, whereas those in the experimental system are more sparsely distributed (approximately 150 monomers apart from rough calculations). However, because the side chains still remain significantly smaller than the molecular pores in the network, the side-chain conformations and their accessibility for mediating external hydrogen bonds are not likely to be affected much by the larger cross-link density used in our simulations. Hence, our “compact” model of the hydrogel network might still yield quantitative information on the conformations of the side chains and their dependence on side-chain length. The intramolecular and intermolecular interactions in the network were described using the ab initio-based polymer consistent force field in Sun H, J Phys Chem B (1998) 102:7338-7364, which is incorporated herein in its entirety. All simulations were performed using the large scale atomic/molecular massively parallel simulator package [http:][lammps.sandia.gov]. Approximately 6,000 water molecules were added and evaluated using the TIP3P model in the unit cells constructed for the networks studied herein. The resulting configurations were energy minimized, equilibrated for 50 ps in the constant-volume-temperature ensemble at 300 K, thermally annealed at 800 K for 40 ps, and then cooled back to 300 K. Further equilibration was performed in the constant-pressure-temperature ensemble at 300 K and 1 atm. The time step was taken as 0.25 fs in all simulations and a Nose-Hoover thermostat was employed to keep temperature constant. The final equilibrated density of equilibrated hydrogels at 300 K and 1 atm was calculated as 1.092 g/cm³ for A6ACA, 1.0744 g/cm³ for A8ACA, and 1.065 g/cm³ for A11AUA groups of the network side chains for mediating hydrogen bonds, five representative configurations of the network from 250-ps-long simulation runs were selected. The accessibility of each group in terms of the number of hydrogen bonds it forms with the water molecules was quantified. The accessibility of the functional groups for interacting with water molecules was assumed to be a good measure for their accessibility for interacting with functional groups from the opposing hydrogel surface. The number of hydrogen bonds mediated by the amide and carboxylic groups of the chosen configurations were calculated by using University of California, San Francisco Chimera software (http)(www.cgl.ucsf.edu/chimera) using a tolerance of 0.3 Å and 20° from the precise geometrical criteria for hydrogen bonding.

Role of Hydrogen Bonding in Self-Healing.

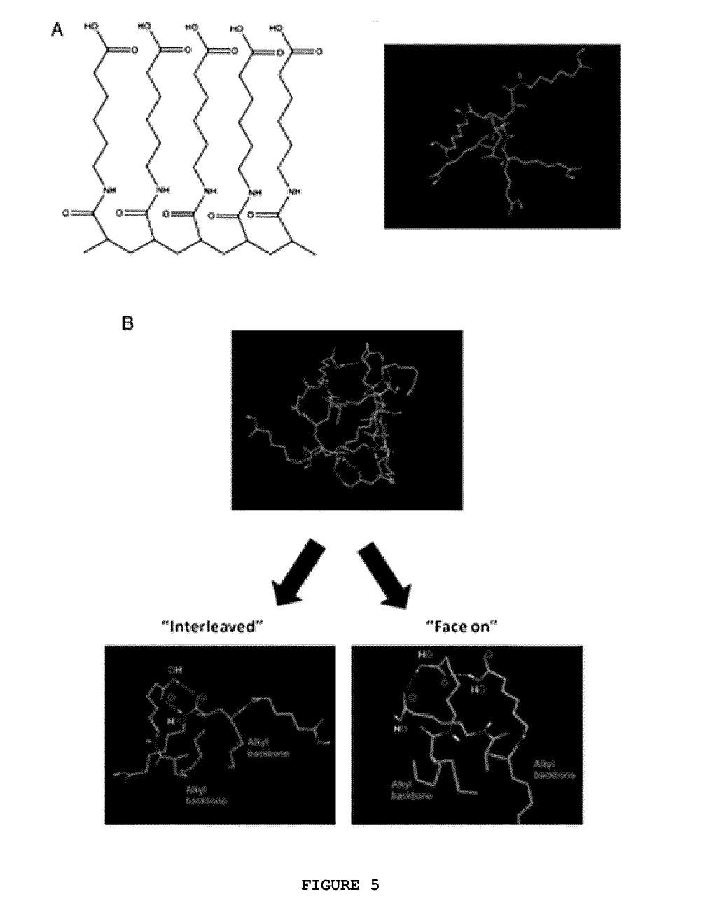

To confirm that the observed healing in A6ACA hydrogels was mediated through hydrogen bonding, the healed hydrogels were immersed into a urea solution. As was expected, the immersion resulted in the separation of the two hydrogels at their interface (see FIG. 2). The role of hydrogen bonding was further analyzed by using FTIR-attenuated total reflectance (ATR) and Raman spectroscopy (see FIGS. 4 A-B, and TABLE 2). The hydrogen-bonded terminal carboxylic-acid group was evident from the Raman band at 1,714 cm⁻¹ and IR band at 1,704 cm⁻¹ observed in healed hydrogels (see FIGS. 4 A-B). The spectroscopic analyses of the healed hydrogels suggested two different types of hydrogen bonding across the interface. First, the spectral features supported direct interaction of carboxyl groups with the amide groups of the opposing pendant side chain in an interleaved configuration (see FIG. 4C). In particular, the prominent IR band at 1,627 cm⁻¹ and the corresponding weak Raman band at 1,624 cm⁻¹, assigned to the amide I mode (majority C═O stretch, some C—N stretch), indicated the presence of strongly hydrogen-bonded amide groups. Second, the spectral features suggested a smaller fraction of carboxyl groups interacting with the opposing carboxyl groups in a face-on configuration (see FIG. 4C). Consistent with this configuration, evidence of a small population of amide groups having similar amide I band intensity for healed and unhealed hydrogels was observed, suggesting similarity in their H-bond environment irrespective of their protonation state. It was confirmed through molecular modeling that the interleaved and face-on configurations were sterically feasible (see FIG. 5). The above analyses suggest an intriguing mechanism for the observed pH-mediated self-healing. At low pH, the terminal carboxyl groups were mostly protonated, which allowed them to form hydrogen bonds with other terminal-carboxyl groups or amide groups across the interface, thereby allowing the hydrogels to weld (see FIG. 4C). At pH above their pKa (4.4 for 6-aminocaproic acid, the parent amino acid from which the A6ACA monomer was synthesized), the A6ACA carboxyl groups were deprotonated and exhibited significant electrostatic repulsion, which prevented hydrogen bonding (see FIG. 4D). It was also found that the healing ability of the hydrogels diminished with prolonged exposure to low-pH environment prior to healing, but could be restored by immersing the hydrogels in a high-pH environment followed by reintroduction into a low-pH environment. The prolonged exposure of the hydrogels to a low-pH environment could lead to intramolecular hydrogen bonding, which decreases their availability to form intermolecular hydrogen bonds across the interface.

Determination of Steric Feasibility of Configurations.

It was determined whether the face-on and interleaved configurations were sterically feasible. As a model system, a five-unit oligomers of A6ACA was used (see FIG. 5A). Two such oligomers were brought together and an energy minimization was performed using ChemBio3D Ultra 12.0 (CambridgeSoft) (see FIG. 5B). Both face-on and interleaved species were observed in the resultant configurations (see FIG. 5B), indicating that both types of configurations are sterically feasible.

Mechanical Characterization of Healed Hydrogels.

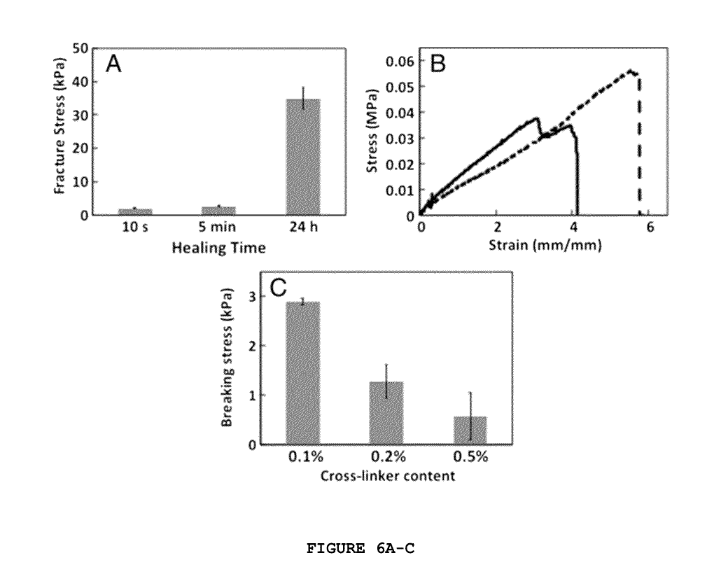

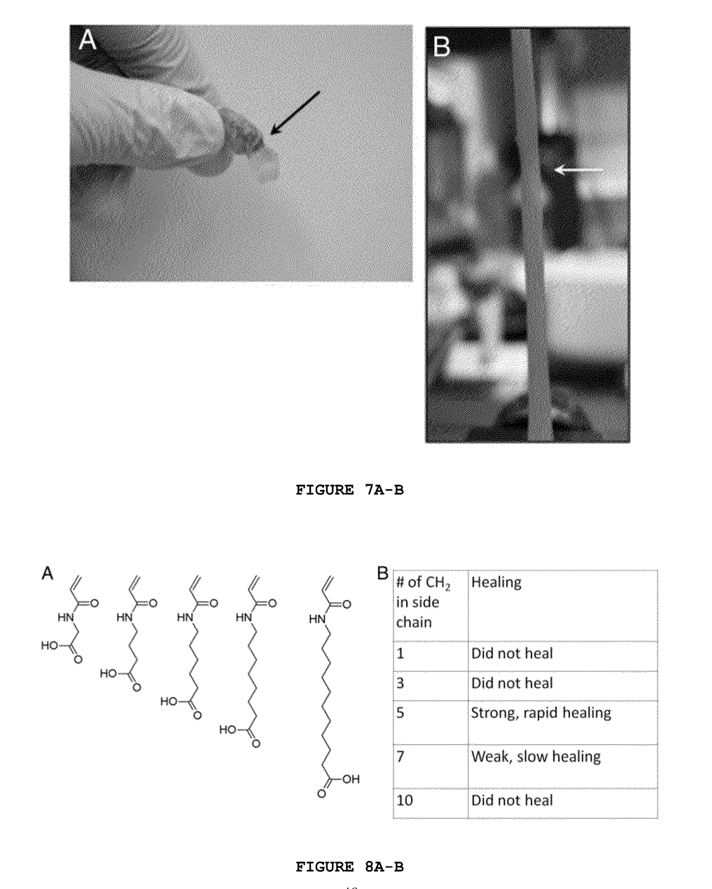

A study of the temporal dependence of the healing indicated an increase in weld-line strength with time over a period of 10 seconds to 24 hours (see FIG. 6A). Hydrogels healed for 10 seconds withstood more than 2.04±0.07 kPa stresses whereas those healed for over 5 min failed upon an application of 2.7±0.2 kPa stress. In both cases, the hydrogels always ruptured in the bulk region, whereas the welded interface remained intact (see FIG. 7A), indicating a strongly healed interface. The low mechanical strength of the bulk region was attributed to its inherent soft nature compared to the surfaces that were in contact with the low-pH solution, as schematically shown in FIG. 4E. Therefore, the interfacial region toughened as a result of protonation of the carboxyl groups and subsequent increase in their hydrogen bonding. In contrast, the interior bulk regions remained soft because protons could not diffuse into the polymer network within the experimental timescales. However, after extended exposure (approximately 24 hours) to low-pH solution, the hydrogels become capable of withstanding large stresses (35±3 kPa) and break at the interface. Moreover, the 24-hours healed hydrogels become opaque because of protonation-induced hydrophobic collapse of the polymer chains (see FIG. 7B). FIG. 6B demonstrated that the maximum stress required to break 24-hour healed hydrogels is 66±7% of that of single hydrogel pieces of similar dimensions treated under identical conditions. The fracture stress in healed hydrogels was lower than in single hydrogels because failure in healed hydrogels involved only breakage of intermolecular hydrogen bonds across the interface whereas failure in single hydrogels involved breakage of both covalent bonds and intramolecular hydrogen bonds. The ratio of the elastic moduli, E_{healed}/E_{single}=1.1±0.5 (where E_{healed} and E_{single} represent the elastic moduli of the 24-hours healed and unhealed hydrogels, respectively) indicated little change in the stiffness of the hydrogels after healing.

Molecular Dynamics Simulations of Hydrogel Networks.

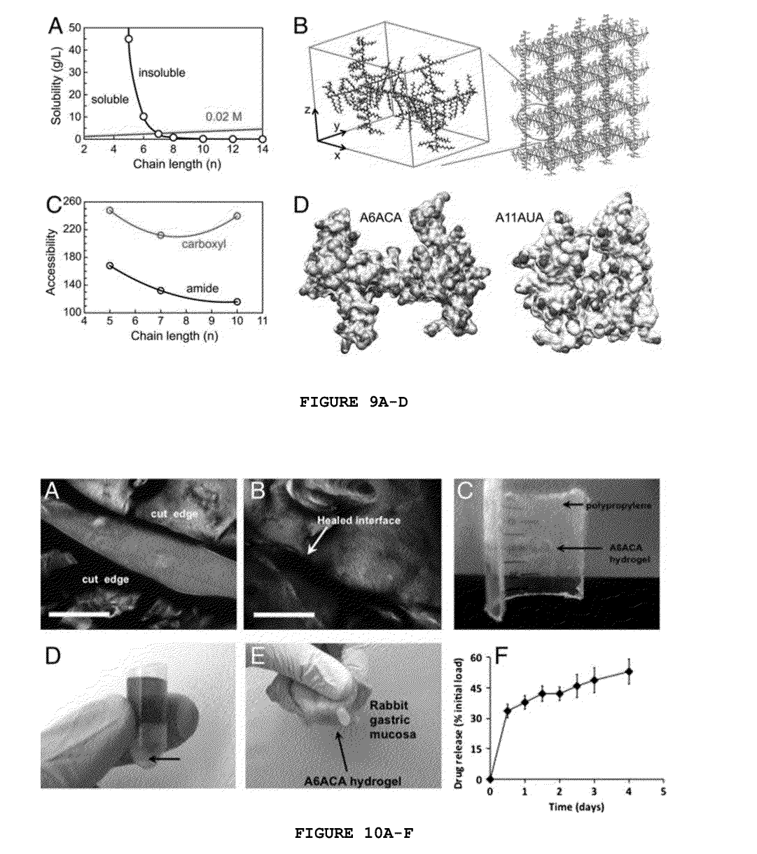

To investigate the effect of side-chain length on healing efficiency, molecular dynamics simulations were performed with hydrogel networks built from A6ACA, A8ACA, and A11AUA monomers having side chains of lengths 5, 7, and 10 CH₂ groups, respectively. A nine-arm hydrogel motif was placed inside the simulation box along with water molecules (see FIG. 9B, Left) and replicated via periodic boundary conditions to yield the desired hydrogel network (see FIG. 9B, Right). A gap in the ±x direction prevented continuity of the network along the x direction and allowed the creation of a hydrogel-water interface in between periodic images of the network. The accessibility of the amide and carboxyl groups was quantified in terms of the number of hydrogen bonds they form with the water molecules. It was assumed that the accessibility of the functional groups for interacting with water molecules is a good measure for their accessibility for interacting with functional groups from the opposing hydrogel surface.

Effect of Cross-Link Density and Side-Chain Length on Healing.

To determine the effect of cross-link density on healing, A6ACA hydrogels with varying cross-linker content were prepared (see FIG. 3C). The self-healing depended strongly on the extent of cross-linking and thereby the swelling behavior of the hydrogels. Specifically, the interfacial strength of healed hydrogels decreased with increasing cross-linker content (see FIG. 6C). The reduction in healing efficiency could be attributed to either the restricted mobility of the side chains or to the decrease in the compliance of the hydrogel with increasing cross-linking, both of which could impede the formation of hydrogen bonds across the interface. The latter effect, however, seems to be the more likely explanation given that the hydrogel still exhibited significant swelling at the high cross-link densities, indicating that the molecular pores might be considerably larger than the side chains and thus do not interfere significantly with the side-chain mobility. Next, the effect of pendant side-chain length on healing was investigated by synthesizing hydrogels with similar cross-linker content but varying side-chain lengths, containing 1-10 methylene groups, terminating with a carboxyl group (see FIG. 8A). Hydrogels with side chains containing 1-3 and 10 methylene groups did not exhibit any healing and those containing 7 methylene groups [N-acryloyl 8-aminocaprylic acid (A8ACA)] demonstrated weak healing (see FIG. 8B). The A8ACA hydrogels required more than 5 minutes to heal, and the healed hydrogels could be separated easily by a small stress (0.267±0.008 kPa). Thus, interestingly, the healing ability depended nonmonotonically on the side-chain length. The low healing ability of hydrogels with short side chains could be attributed to the limited “reach” of the carboxyl groups in mediating hydrogen bonds with functional groups across the interface, especially given that the hydrogel surfaces are likely corrugated. As the side chains become longer, the terminal carboxyl groups become more flexible and increase their reach for hydrogen bonding, especially with the internal amide groups of the opposing hydrogel. When the side chains become too long, they begin to pose a larger steric hindrance to the interactions between the carboxyl and amide groups. In addition, the long side chains tend to aggregate and collapse because of increased hydrophobic interactions. This effect can be gleaned from the water solubility of carboxylic acids of varying hydrocarbon chain lengths (see FIG. 9A); i.e., chains containing more than six CH₂ groups become insoluble in water at concentrations similar to the effective concentration of side chains present in the hydrogel (approximately 0.02 M for A6ACA). Both the steric hindrance and hydrophobic collapse reduce the accessibility of the amide groups, leading to a reduction in the healing efficiency. To confirm the suggested decrease in the accessibility of the amide groups with increasing chain length, molecular dynamics simulations of A6ACA, A8ACA, and N-acryloyl 11-aminoundecanoic acid (A11AUA) hydrogel networks in an aqueous medium were conducted (see FIG. 9B). The accessibility of the terminal-carboxyl and internal-amide groups in terms of the average number of hydrogen bonds they form with the surrounding water molecules during the simulation was quantified (see FIG. 9C). The simulations demonstrate a substantial decrease in the accessibility of the amide groups with increasing side-chain length, whereas the accessibility of the carboxyl groups changes only slightly with the chain length. FIG. 9D provides representative configurations of the A6ACA and A11AUA hydrogel within one unit cell obtained from the simulations. The configurations are shown in a solvent excluded surface representation to illustrate the reduction in the accessibility of the amide groups (shown in blue) in going from the short to long side chains.

The correlation between amide group accessibility and healing ability for A6ACA, A8ACA, and A11AUA hydrogel provided further support for the dominant role played by the interleaved hydrogen bonding configuration in self-healing as evidenced from spectroscopic analyses. The observed dependence of healing on the side-chain length thus confirms that self-healing is best exhibited by hydrogels possessing a balance of hydrophobic and hydrophilic interactions. Interestingly, this requirement along with that for flexible side chains to mediate hydrogen bonding across the interface explains why many polymeric systems including protein hydrogels do not exhibit robust self-healing despite their possessing amide and carboxylic functional groups.

A6ACA Hydrogels as Self-Healing Coating.

A6ACA hydrogels were swollen in a 0.01% solution of methyl red in PBS, to gain contrast between the coating and the surface. Polystyrene surfaces were coated with the hydrogels by drying at 37° C. for 12 hours. A 300-μm-wide scratch was made in the coating surface using a surgical scalpel and imaged using bright field microscopy (Axio Observer A1; Carl Zeiss) (see FIG. 10A). The scratch site was briefly hydrated for 60 seconds with 50 μL DI water after which the excess water was removed and the site was treated with 100 μL of 0.1 M HCl. The cut edges facing each other were then reimaged after 5 minutes (see FIG. 10B).

Adhesion of A6ACA Hydrogels to Plastics.

A6ACA hydrogels were swollen in PBS for 4 hours. The swollen hydrogel was found to adhere to polypropylene and polystyrene surfaces within 15-20 seconds upon spraying with pH-0.3 solution at the hydrogel-plastic interface (see FIG. 10C).

A6ACA Hydrogels for Sealing Acid Leakages.

The conical bottom portion of a 2-mL centrifuge tube (Eppendorf) was cut out to create a hole, measuring approximately 1 cm in diameter. The hole was plugged using PBS-swollen A6ACA hydrogels. This sealed conical tube was then filled with 1 mL of 0.5 M HCl (with 0.5% added methyl red, to make the solution pink for ease of visualization) and photographed to show lack of any leak (see FIG. 10D).

A6ACA Hydrogels as Mucoadhesive Polymer.

Stomach tissues were resected from freshly killed New Zealand white rabbits and carefully rinsed with PBS to remove residual food material. After cleaning, the tissues were maintained in PBS and used for the experiments the same day. To investigate mucoadhesiveness of A6ACA hydrogels, hydrogels were first maintained in contact with inner gastric lining under immersion in simulated gastric acid [HCl—KCl buffer of pH 1.5 containing 54.7% (by volume) 0.2 M KCl and 45.3% 0.2 M HCl] for 20 minutes and then photographed (see FIG. 10E).

A6ACA Hydrogel as a Drug Carrier.

A solution containing 0.5 mg/mL tetracycline (50×) in PBS was prepared from a stock solution of tetracycline (1,000×, 10 mg/mL in 70% ethanol). As-synthesized A6ACA hydrogels (n=4) were loaded with tetracycline by placing them in this solution for 24 hours. Based on the known swelling ratio of A6ACA hydrogels in PBS, the total tetracycline load was calculated for each hydrogel. The hydrogels were then immersed in 40 mL of simulated gastric fluid (pH 1.5) and placed on a shaker at 150 rpm. Every 12 hours, 4 mL of the immersion solutions were collected and replaced with 4 mL of fresh buffer. The released tetracycline in the collected solutions was measured spectrophotometrically at 270 nm. The total tetracycline release (expressed as percentage of total tetracycline load, calculated from swelling ratio of hydrogels) was calculated for each time point and averaged across the replicates (see FIG. 10F).

A number of embodiments have been described herein. Nevertheless, it will be understood that various modifications may be made without departing from the spirit and scope of this disclosure. Accordingly, other embodiments are within the scope of the following claims.

What is claimed is:

1. A self-healing hydrogel that comprises one or more cross-linking precursors and one or more polymer precursors comprising a pendant side-chain of Formula I: 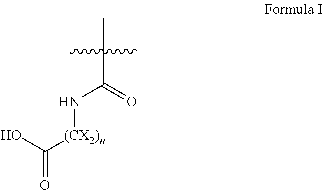
wherein,
n is an integer from 1 to 10;
each X is independently selected from H, D, optionally substituted (C₁₋₆)-alkyl, optionally substituted (C₁₋₆)-heteroalkyl, optionally substituted (C₁₋₆)-alkenyl, optionally substituted (C₁₋₆)-heteroalkenyl, optionally substituted (C₁₋₆)-alkynyl, optionally substituted (C₁₋₆)-heteroalkynyl, optionally substituted cylcoalkyl, optionally substituted cycicoalkenyl, halide, alcohol, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester.

2. The self-healing hydrogel of claim 1, wherein one or more polymer precursors comprise a pendant side-chain of Formula I: 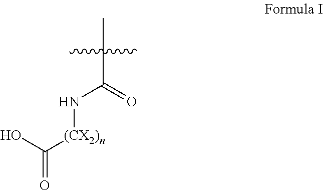
wherein,
n is an integer from 1 to 11;
each X is independently selected from H, D, and optionally substituted (C₁₋₆)-alkyl.

3. The self-healing hydrogel of claim 1, wherein the one or more polymer precursors comprise a pendant side-chain of Formula I(a):

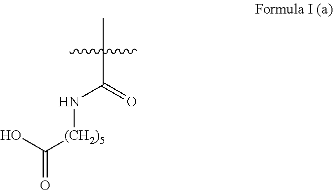

4. The self-healing hydrogel of claim 1, wherein the one or more cross-linking precursors are selected from the group consisting of optionally substituted N,N′-methylenebisacrylamide, 1,4-cyclohexanedimethanol divinyl ether, ethylene glycol diacrylate, ethylene glycol dimethacrylate, divinylbenzene, 4,4′-methylenebis(cyclohexyl isocyanate), 1,6-hexanediol diacrylate, 1,4-phenylenediacryloyl chloride, and tetra(ethylene glycol) diacrylate.

5. The self-healing hydrogel of claim 1, wherein the cross linking precursor is N,N′-methylenebisacrylamide.

6. The self-healing hydrogel of claim 1, wherein the hydrogel comprises 0.01% to 1% percent of cross-linking precursors.

7. The self-healing hydrogel of claim 1, wherein the hydrogel comprises about 0.1% of cross-linking precursors.

8. The self-healing hydrogel of claim 1, wherein the pendant side chain can form at least two hydrogen bonds to one or more additional pendant side chains.

9. The self-healing hydrogel of claim 8, wherein the hydrogen bonds can form when the hydrogel is exposed to a pH of less than or equal to 5.

10. The self-healing hydrogel of claim 8, wherein the hydrogen bonds break when the hydrogel is exposed to a pH of greater than or equal to 9.

11. A structure comprising at least two or more hydrogels of claim 1 which are linked together by hydrogen bonding.

12. A self-healing coating comprising a hydrogel of claim 1.

13. A self-healing sealant comprising a hydrogel of claim 1.

14. A tissue adhesive comprising a hydrogel of claim 1.

15. The tissue adhesive of claim 14, wherein the tissue adhesive is used as a mucoadhesive for gastric tissue.

16. A drug carrier comprising a hydrogel of claim 1.

17. The drug carrier of claim 16, wherein the drug carrier controllable releases one or more pharmaceutical agents in the gastrointestinal tract of a subject.
